(19) United States
(12) Patent Application Publication
(10) Pub. No.: US 2005/0043615 A1
(43) Pub. Date: Feb. 24, 2005

(54) Portable, low-profile integrated computer, screen and keyboard for computer surgery applications

(76) Inventors: Gary Shigeru Natsumi, Brooklyn, NY (US); Rony A. Abovitz, Hollywood, FL (US); Michelle Zaller, Medina, OH (US)

Correspondence Address: MUNSCH, HARDT, KOPF & HARR, P.C., INTELLECTUAL PROPERTY DOCKET CLERK, 1445 ROSS AVENUE, SUITE 4000, DALLAS, TX, 75202-2790, US

(21) Appl. No.: 10/772,137

(22) Filed: Feb. 4, 2004

Related U.S. Application Data
(60) Provisional application No. 60/319,924, filed on Feb. 4, 2003.
(60) Provisional application No. 60/444,824, filed on Feb. 4, 2003.
(60) Provisional application No. 60/444,975, filed on Feb. 4, 2003.
(60) Provisional application No. 60/445,078, filed on Feb. 4, 2003.
(60) Provisional application No. 60/444,989, filed on Feb. 4, 2003.
(60) Provisional application No. 60/444,988, filed on Feb. 4, 2003.
(60) Provisional application No. 60/445,002, filed on Feb. 4, 2003.
(60) Provisional application No. 60/445,001, filed on Feb. 4, 2003.

(51) Int. Cl. A61B 5/05

(52) U.S. Cl. 600/429

(57) ABSTRACT

An integrated computer, a display device and a keyboard for computer surgery applications is disclosed. The display device is coupled to a computer case by a bracket. One end of the bracket is coupled to the computer case by a hinge and the other end of the bracket is coupled to a back surface of the display device by another hinge.

This patent application claims the benefit of U.S. provisional patent application Ser. No. 60/319,924, entitled “Portable, Low-Profile Integrated Computer, Screen and Keyboard for Computer Surgery Applications,” filed Feb. 4, 2003, the disclosure of which is incorporated herein by reference. This application relates to the following United States provisional patent applications: Ser. No. 60/444,824, entitled “Interactive Computer-Assisted Surgery System and Method”; Ser. No. 60/444,975, entitled “System and Method for Providing Computer Assistance With Spinal Fixation Procedures”; Ser. No. 60/445,078, entitled “Computer-Assisted Knee Replacement Apparatus and Method”; Ser. No. 60/444,989, entitled “Computer-Assisted External Fixation Apparatus and Method”; Ser. No. 60/444,988, entitled “Computer-Assisted Knee Replacement Apparatus and Method”; Ser. No. 60/445,002, entitled “Method and Apparatus for Computer Assistance With Total Hip Replacement Procedure”; Ser. No. 60/445,001, entitled “Method and Apparatus for Computer Assistance With Intramedullary Nail Procedure”; each of which was filed on Feb. 4, 2003 and is incorporated herein by reference. This application also relates to the following applications filed concurrently herewith: Attorney Docket No. 4204.29-1, entitled “Interactive Computer-Assisted Surgery System and Method”; Attorney Docket No. 4204.30-1, entitled “System and Method for Providing Computer Assistance With Spinal Fixation Procedures”; Attorney Docket No. 4204.31-1, entitled “Computer-Assisted Knee Replacement Apparatus and Method”; Attorney Docket No. 4204.32-1, entitled Computer-Assisted External Fixation Apparatus and Method”; Attorney Docket No. 4204.33-1, entitled “Computer-Assisted Knee Replacement Apparatus and Method”; Attorney Docket No. 4204.34-1, entitled “Method and Apparatus for Computer Assistance With Total Hip Replacement Procedure”; and Attorney Docket No. 4204.35-1, entitled “Method and Apparatus for Computer Assistance With Intramedullary Nail Procedure”; all of which are incorporated herein by reference.

### TECHNICAL FIELD OF THE INVENTION

The present invention relates generally to a computer system for surgery applications.

### BACKGROUND OF THE INVENTION

Computer-based surgical navigation systems provide real time during surgery and interventional procedures. These objects may include markers placed on a patient, surgical instruments, implants, and other objects used in such procedures. Image-based systems, a pre-operative or intra-operative diagnostic image data set (2 dimensional or 3 dimensional) is used as a reference or map. Such systems indicate the actual position and/or orientation of, for example, a surgical object to a patient's anatomy with reference to diagnostic images of the patient's anatomy. “Image-less” systems—meaning without use of a patient's diagnostic images—may also be used. Some systems may also be used without diagnostic images, for example to track tools with respect to anatomical features of the patent that were localized without use of diagnostic image. The computer-assisted navigation system may be combined with a robotic arm or similar mechanism.

Computer assisted surgical navigation systems include a computer, which executes software that performs the various functions of the system, coupled to a localizer or tracking system that tracks one or more objects. Typically, the computer must be placed on a cart. Often, a monitor or display is mounted on the cart on an articulating arm that is mounted to the cart. In either case, the system is relatively large and isn't easily transportable. It must be rolled from room to room. Furthermore, in a crowded operating room, it can get in the way of use of other equipment.

### SUMMARY OF THE INVENTION

In accordance with an embodiment of the present invention, a compact, integrated case housing a computer for assisting with surgical and interventional procedures is integrated with a display device and a keyboard. The integrated device can be folded into a relatively compact unit for storage or transport when not in use. When in use, the position of the display device with respect to the computer case can be adjusted manually for viewing.

In the example of a preferred embodiment of the invention illustrated in the accompanying figures, the display device is coupled to a computer case by a bracket. One end of the bracket is coupled to the computer case by a hinge and the other end of the bracket is coupled to a back surface of the display device by another hinge. The display device may be pivoted and folded, with the screen facing the computer case, for storage and transport. The display device may also be pivoted so that it faces away from the case, and its position adjusted up and down and tilted for the most desirable orientation during surgery.

Other aspects and features of the invention will become apparent to those ordinarily skilled in the art upon review of the following description of specific embodiments of the invention in conjunction with the accompanying figures.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is a perspective view of a computer in a closed configuration in accordance with an embodiment of the present invention;

FIG. 2 is a perspective view of a computer in an open configuration in accordance with an embodiment of the present invention;

FIG. 3 is a perspective view of the computer of FIG. 2 mounted to a stand;

FIG. 4 is a perspective view of a computer mounted to a stand with the computer in an operating configuration;

FIG. 5 is a perspective view of the computer of FIG. 1 mounted to a stand;

FIG. 6 is a perspective view of a stand in accordance with an alternative embodiment of the present invention; and

FIG. 7 is a perspective view showing the side and front of the computer of FIG. 1 mounted to a stand.

### DETAILED DESCRIPTION OF THE DRAWINGS

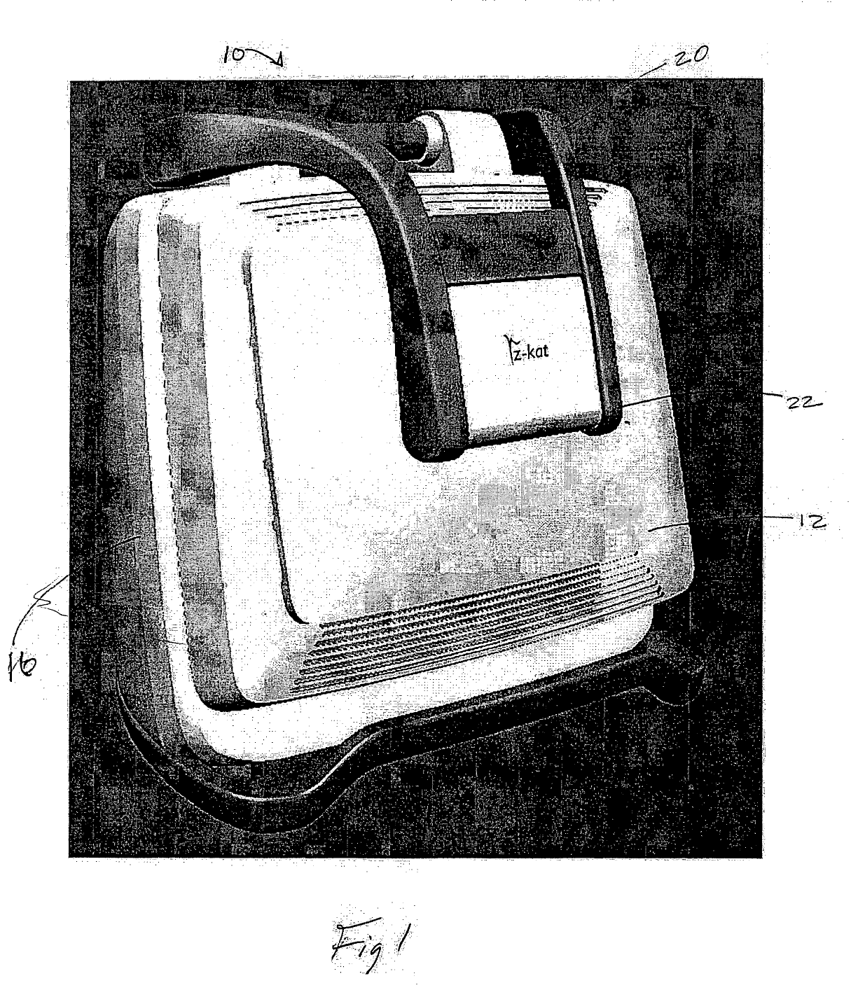

With reference to FIGS. 1-7, a computer 10 includes a flat visual display screen 12, keyboard 14 (shown only in a stowed position) and electronics case or enclosure 16.

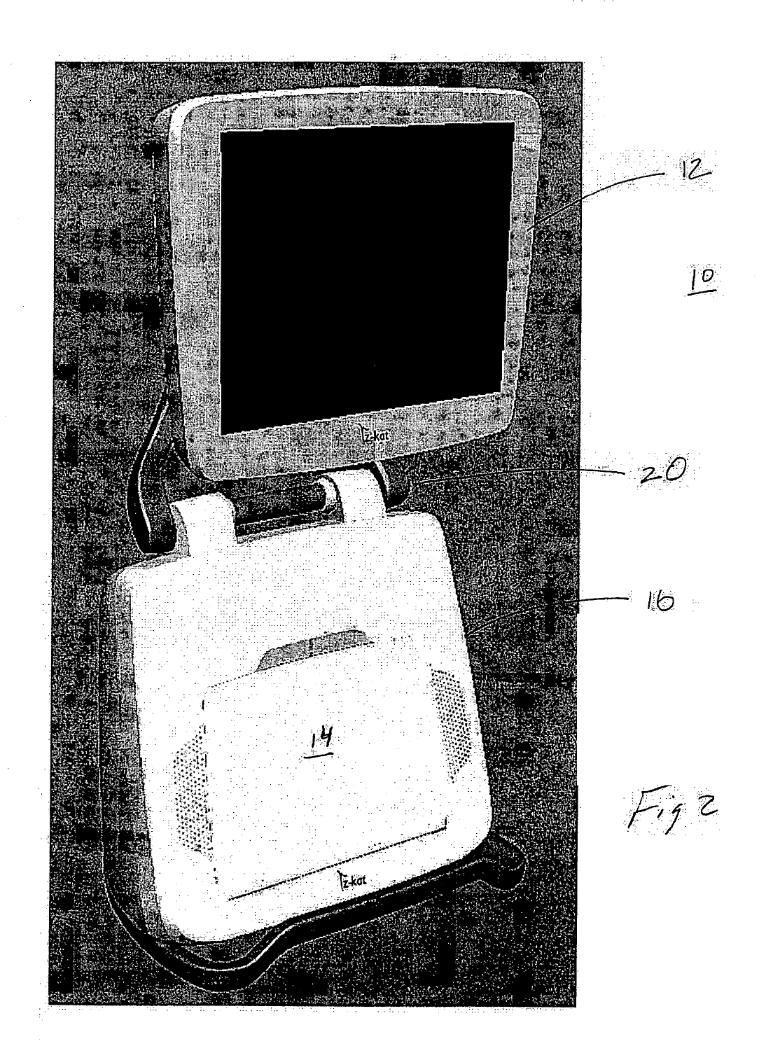

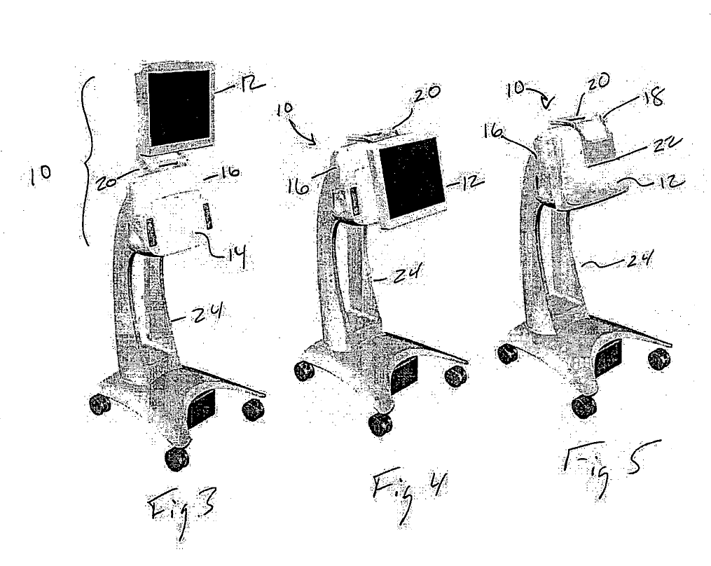

Bracket 18 is coupled to a rear surface of visual display screen 12 by a hinge 22 and to a top surface of enclosure 16 by hinge 20. This permits the visual display to be folded up from a stowed position shown in FIGS. 1 and 5 up to a fully extended position shown in FIGS. 2 and 3. It also permits the display screen to be lowered from the full extended position to a lower position as shown in FIG. 4 while facing in the same direction. It is therefore well suited to an application such as surgery, during which the users of the computer are likely standing. It can be adjusted to a comfortable height for the surgeon.

Keyboard 14 can be folded down if it is needed during set up, but folded up while the computer is being used during surgery.

The computer is mounted to a stand 24 or 26 when it is used in surgery. For transport or storage, it can be removed and stowed. The keyboard is folded into the enclosure and the screen is folded down so that it is adjacent the enclosure and protected. Hinge 20 also preferably functions as a handle.

FIG. 1 is a perspective view of computer 10 in a closed configuration in which the keyboard and the visual display are in a stowed position. FIG. 2 is a perspective view of computer 10 in an open configuration in which display screen 12 is in a fully extended position and keyboard 14 is in a stowed position. FIG. 3 is a perspective view of computer 10 in which display screen 12 is in a fully extended position, keyboard 14 is in a stowed position and computer 10 is mounted to stand 24. FIG. 4 is a perspective view of computer 10 in an operating configuration in which display screen 12 is in a lower position while facing substantially the same direction as in the open configuration of FIG. 2, and computer 10 is mounted to stand 24. FIG. 5 is a perspective view of computer 10 mounted to stand 24 in a closed configuration in which display screen 12 and keyboard 14 are both in a stowed position.

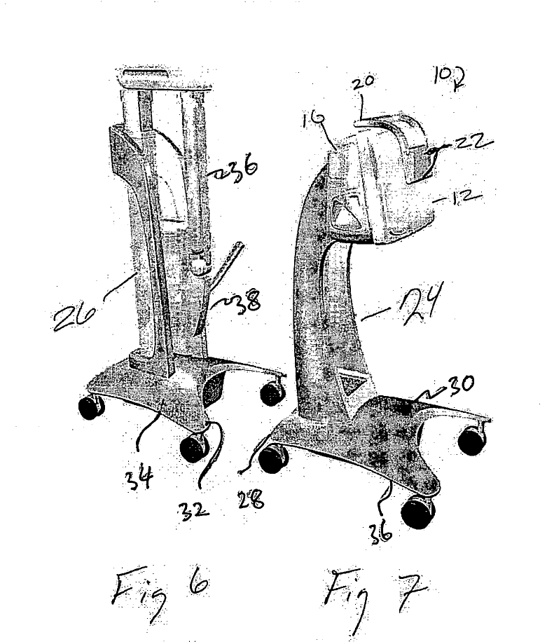

FIG. 6 is a perspective view of a stand 26 in accordance with an alternative embodiment of the present invention and FIG. 7 is a perspective view of computer 10 mounted to stand 24 in the closed configuration. The rear portion 28 of base 30 of stand 24 is narrower than the front portion 32 of base 34 of stand 26. This enables stand 24 to be removably interlocked or nested with stand 26. Rear portion 28 of base 30 is also narrower than front portion 36 of base 30. Stand 26 comprises a pivotable arm 36 to which a camera pair 38 may be coupled. Because rear portion 28 of base 30 is narrower than front portion 32 of base 34, stand 24 may be removably interlocked with stand 26 by inserting rear portion 28 of base 30 into front portion 32 of base 34. Interlocking of stands 24 and 26 enables the camera pair and computer to be stored away together. If desired, the stands may also be interlocked during surgery.

While the invention has been particularly shown and described by the foregoing detailed description, it will be understood by those skilled in the art that various other changes in form and detail may be made without departing from the spirit and scope of the invention.

1. An integrated computer and display for use in a computer-assisted surgical or interventional procedures, comprising:
a case housing a computer for running a computer-assisted surgical navigation process;
a panel-shaped display device having a screen on one side;
a bracket operable to couple said display device to said case, whereby the display is pivotable between a stowed position in which the screen faces a side of the case and an open position, in which the screen faces away from the case.

2. The computer of claim 1, further comprising a first hinge to hingeably couple a first end of said bracket to said case.

3. The computer of claim 2, further comprising a second hinge to hingeably couple a second end of said bracket distal from said first end to a rear surface, opposite the screen, of said display device.

4. The computer of claim 1, further comprising a keyboard hingeably coupled to said case.

5. A surgery system, comprising:
a stand; and
an integrated computer couplable to said stand, said integrated computer comprising:
a case;
a display device coupled to said case; and
a bracket operable to couple said display device to said case, said bracket further operable to facilitate pivotal movement of said display device from a fully extended position to a lowered position.

6. The system of claim 4, wherein said stand comprises a base having a front end and a rear end, said rear end of said stand being narrower than said front end.
